(19) United States
(12) Patent Application Publication
(10) Pub. No.: US 2025/0299324 A1
(43) Pub. Date: Sep. 25, 2025

(54) MACHINE LEARNING-BASED IMAGE PROCESSOR FOR INTRAORAL IMAGES

(71) Applicant: The University of Hong Kong, Hong Kong (HK)

(72) Inventors: Yu Hang LAM, Hong Kong (HK); Richard Tai Chiu HSUNG, Hong Kong (HK); Andrew Chi Chung CHENG, Hong Kong (HK); Guan-Hua LI, Shenzhen (CN); Wai-Lun LO, Hong Kong (HK); Chun Wang CHAU, Hong Kong (HK); Khaing Myat THU, Hong Kong (HK)

(21) Appl. No.: 18/608,983

(22) Filed: Mar. 19, 2024

(51) Int. Cl. G06T 7/00; G06N 20/00; G06T 7/11; G16H 30/20; G16H 30/40

(52) U.S. Cl. CPC G06T 7/0012; G06N 20/00; G06T 7/11; G16H 30/20; G16H 30/40; G06T 2207/30036

(57) ABSTRACT

A machine learning (ML) based image processing system for image processing of intraoral images is provided. The system includes an image receiver dedicated to processing frontal view intraoral images, an object detector for filtering and identifying regions within these images, and the segmentation of these regions into pixel blocks. Employing multiple autoencoders with deep neural networks, the system conducts image analysis and annotation, generating markings for oral structure conditions. These markings are then seamlessly integrated into a comprehensive marking through an ensemble integrator.

### FIELD OF THE INVENTION

The present invention generally relates to the machine-learning based image processors. More specifically the present invention relates to machine learning (ML) based image processors for processing intraoral structure images.

### BACKGROUND OF THE INVENTION

Periodontal disease, encompassing gingivitis and periodontitis with reversible and irreversible tissue damage, presents a significant global health burden, accounting for 21% of productivity loss (USD 38.85 billion). Prevalence exceeds 50% worldwide, with one-third classified as severe cases. Gingivitis is reversible, but periodontitis requires interventions. The disease exhibits site-specific variations, necessitating thorough clinical site evaluations for effective care. Traditional methods rely on visual assessment by dentists, lacking acute symptoms for patient awareness. Despite significant efforts in oral hygiene reinforcement, plaque control remains challenging.

In a broader context, oral health is integral to overall well-being, especially for vulnerable populations like older adults. The aging demographic faces increasing prevalence of oral diseases, impacting oral health-related quality of life (OHRQoL) and serving as systemic disease risk factors. Access to oral healthcare is limited for older adults due to various barriers, including economic constraints, lack of infrastructure, and cognitive decline. Traditional dental care models are insufficient, prompting exploration of technological solutions.

Artificial intelligence (AI) emerges as a potential solution. AI, as a core focus, integrates into oral healthcare systems to enhance accessibility, preventive dentistry, and consultation quality. Defined by its ability to mimic human intelligence, AI, notably cognitive computing (CC), aligns human-machine functioning for better decision-making and efficient data processing. Leveraging mobile health (mHealth) systems with AI algorithms enables remote monitoring and diagnosis, extending dental professionals' reach to communities and care homes. The advancement of generative AI (GenAI) introduces the possibility of AI-driven oral care recommendations without direct dentist involvement, empowering older adults to proactively manage their oral health.

AI's role spans from radiography analysis to gingivitis detection in intraoral images. For instance, in an AI application, Dentistry 4.0, including automated digital dentistry, offer diverse clinical benefits. Furthermore, there are several network architectures that are currently used to detect gingivitis from intraoral images with accuracy ranging from 0.47 to 0.83, with 1.00 as the highest accuracy. However, the accuracy for clinical use should be as high as possible, and accuracy of 0.90 or above should be targeted for clinical use, and it is clear that current evaluations lack consensus on accuracy standards. Namely, there's a need for high accuracy in AI systems for predicting gingivitis, particularly in sensitivity and specificity.

Therefore, the present invention addresses this need by introducing a high-precision machine learning based image processing system for image processing of intraoral images, with the primary objective of reducing the time that dentists invest in diagnosis and evaluation. Moreover, it opens avenues for patients to conduct self-examinations, contributing significantly to heightened awareness of oral hygiene among the general populace.

### SUMMARY OF THE INVENTION

It is an objective of the present invention to provide systems, or method to solve the aforementioned technical problems.

In accordance with a first aspect of the present invention, a machine learning (ML) based image processing system for image processing of intraoral images is provided. Particularly, the system includes an image receiver for processing one or more frontal view intraoral images of intraoral structures; an object detector that filters frontal view intraoral images, identifies regions for further image processing in the frontal view intraoral images, and segment the regions into pixel blocks; multiple autoencoders with multiple deep neural networks for image analysis and image annotation of the regions and the segmented pixel blocks for generating oral structure condition markings; and an ensemble integrator that integrates the oral structure condition markings to a complete marking.

In accordance with one embodiment of the present invention, the multiple autoencoders using deep neural networks corrects intraoral images by image analysis that corrects the frontal view intraoral images containing blurriness caused by body movement or scaling and performs image color balance.

In accordance with another embodiment of the present invention, the segmentation of the regions of interest is able to be performed multiple times to generate different sets of the pixel blocks in different sizes, so as to increase the accuracy of the marking.

In accordance with one embodiment of the present invention, the frontal view intraoral photographs show at least 3 mm gingival tissue from maxillary and mandibular gingival margins and a clear gingival margin between the gum and teeth.

In accordance with one embodiment of the present invention, the system further includes a user interface and a display for displaying the complete marking.

In accordance with one embodiment of the present invention, the system further includes a memory unit for storing a database of annotated intraoral images, where the multiple autoencoders are trained on this database to improve the accuracy and efficiency of the image analysis.

In accordance with a second aspect of the present invention, a method of processing intraoral images utilizing the aforementioned ML based image processing system is introduced. Specifically, the method includes the following steps: acquiring one or more frontal view intraoral images of intraoral structures by the image receiver; filtering the frontal view intraoral images and identifying regions thereof for further image processing in the frontal view intraoral images utilizing the object detector; segmenting the regions into pixel blocks by the object detector; analyzing and annotating the regions and the segmented pixel blocks utilizing the multiple autoencoders with multiple deep neural networks for generating structure condition markings; and integrating the oral structure condition markings to a complete marking by the ensemble integrator.

In accordance with one embodiment of the present invention, the complete marking includes annotations of a healthy gingival margin, a questionable gingival margin and a diseased gingival margin marked on the frontal view intraoral images.

In accordance with one embodiment of the present invention, the step of analyzing and annotating the regions and the segmented pixel blocks involves analyzing parameters including as the colors of the gum, the smoothness of the gingival margin, the curvature of the gingival margin, and texture features near the gingival margin such as stippling, swelling appearances.

In accordance with one embodiment of the present invention, the healthy gingival margin indicates that the gum is pink, the gingival margin is smooth and there is no bleeding spot on the gum; the questionable gingival margin represents that the gum turns red, the gingival margin is rough or the gum is swollen; and the diseased gingival margin indicates that there are white/red patches on the gum, the gum is generalized redness, there is ulcer on the gum, the gum is swollen or there is bleeding spot on the gum.

In accordance with one embodiment of the present invention, the multiple autoencoders with multiple deep neural networks are trained with standard frontal view intraoral photographs assessed by at least one qualified dentist, wherein the qualified dentist marks the gingival margins displayed in the standard frontal view intraoral photographs and denotes them as healthy, questionable and diseased.

In accordance with another embodiment of the present invention, the frontal view intraoral images show at least 3 mm gingival tissue from maxillary and mandibular gingival margins and a clear gingival margin between the gum and teeth.

### BRIEF DESCRIPTION OF THE DRAWINGS

Embodiments of the invention are described in more details hereinafter with reference to the drawings, in which:

FIG. 1 depicts a ML based image processing system for image processing of intraoral images;

FIG. 2 depicts a schematic diagram showing the system architecture;

FIG. 3 depicts a flow diagram of the data processing; and

FIG. 4 depicts the marking results of the validation set using the adopted segmentation model, in which column A shows the input intraoral image, column B shows the ground truth (health status) labelled by calibrated dentist, and column C displays the marking results; the light grey marking indicates healthy, the white marking means questionable, and the dark grey marking indicates diseased.

### DETAILED DESCRIPTION

In the following description, systems and/or methods of gingivitis detection and/or diagnose and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Periodontal disease is caused by accumulation of plaque biofilm along the gingival margin, resulting in localized gingival inflammation and host responses. An early stage of periodontal disease, gingivitis, may be reversed by removal of plaque, and the progress to later stages of periodontitis may be halted.

The development of periodontal disease is not consistent amongst all teeth and sites, and site predilections, i.e., site-specific, have been observed. For proper self-care or professional care, understanding and evaluations of clinical signs of individual sites are crucial. The clinical signs of gingivitis are inflammation-related and are a result of host response to dental plaque. As inflammation occurs at the gingival margin, redness (ie, change in colour); swelling (ie, change in volume); and loss of stippling appearance as loss of gingival fiber attachment (ie, change in surface characteristics) are observed, due to increase in blood flow (redness) and leakage of tissue fluid from blood vessels into the tissues (swelling). These changes are generally assessed visually by dentists, and patients may not be aware of the disease progression due to its chronic nature and lack of acute symptoms.

Therefore, the objective of the present invention is to provide systems and/or methods utilizable for marking gingivitis areas in provided intraoral images with accuracy at or above 0.90.

In accordance with a first aspect of the present invention, a machine learning (ML) based image processing system for image processing of intraoral images is provided.

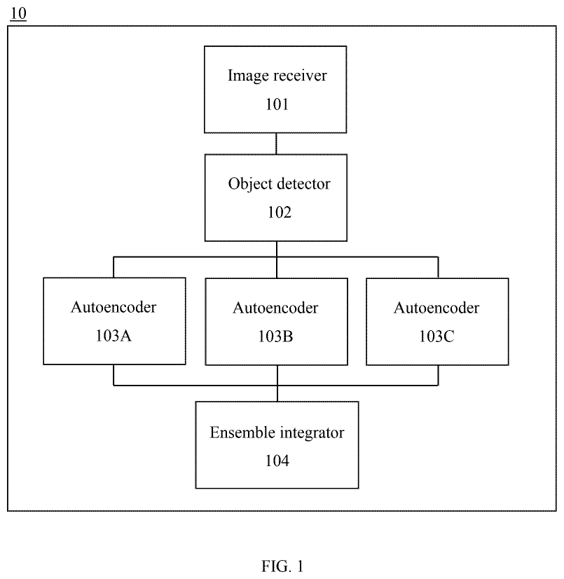

The system encompasses several components working in tandem to achieve accurate and detailed oral structure condition markings. As shown in FIG. 1, a ML based image processing system 10 for image processing of intraoral images in one embodiment of the present invention is depicted. The system 10 incorporates an image receiver 101 responsible for processing one or more frontal view intraoral images of intraoral structures. An integral component is the object detector 102, a mechanism adept at filtering frontal view intraoral images, identifying regions for further analysis, and segmenting these regions into pixel blocks. Multiple autoencoders 103A, 103B and 103C, driven by deep neural networks, play a pivotal role in image analysis and annotation of the segmented pixel blocks, ultimately generating oral structure condition markings. The ensemble integrator 104 serves as the final piece, seamlessly integrating these markings into a complete marking.

It is worth noting that the quantity of the autoencoders can be adjusted based on specific requirements.

This system's autoencoders are highlighted for their proficiency in correcting intraoral images. Leveraging deep neural networks, they conduct image analysis, rectifying blurriness induced by body movement or scaling and ensuring optimal image color balance.

In some embodiments, the autoencoders forms a robust framework for the analysis and annotation of the identified regions of interest and the segmented pixel blocks. This multifaceted approach allows the system to generate oral structure condition markings, capturing the intricacies of gingival health with unparalleled accuracy. The ensemble integrator orchestrates the integration of these markings, culminating in a comprehensive and detailed assessment-a complete marking that precisely marks gingivitis areas on the subject's frontal view intraoral images.

It is important to highlight that the segmentation of regions of interest can be performed iteratively, generating diverse sets of pixel blocks in different sizes. This iterative approach significantly enhances the accuracy of the generated markings.

Furthermore, these frontal view intraoral images distinctly showcase at least 3 mm of gingival tissue from maxillary and mandibular gingival margins, along with a clear gingival margin between the gum and teeth.

In some embodiments, a user interface and display are introduced as integral components of the system. These elements facilitate the visualization of the complete oral structure condition markings, providing a user-friendly interface for healthcare professionals.

In an alternative embodiment, a memory unit may be equipped within the system. This unit is dedicated to storing a database of annotated intraoral images. The multiple autoencoders in the system are trained on this comprehensive database, enhancing their ability to conduct precise and efficient image analysis.

In one embodiment, DeepLabv3+ built on Keras (v2.12, Google LLC) with TensorFlow 2 (v2.9, Google LLC) is adopted. This neural network is highly transferable and offered multiple pre-trained checkpoints to facilitate learning of the datasets. Xception (v1.0, Google LLC) and MobileNetV2 (v1.0, Google LLC) are adopted as the backbone. Xception models use depth-wise separable convolutions with fewer connections and lighter model (ie, faster), and MobileNet models utilize the same convolutions with smaller model size and complexity, making it easier to construct.

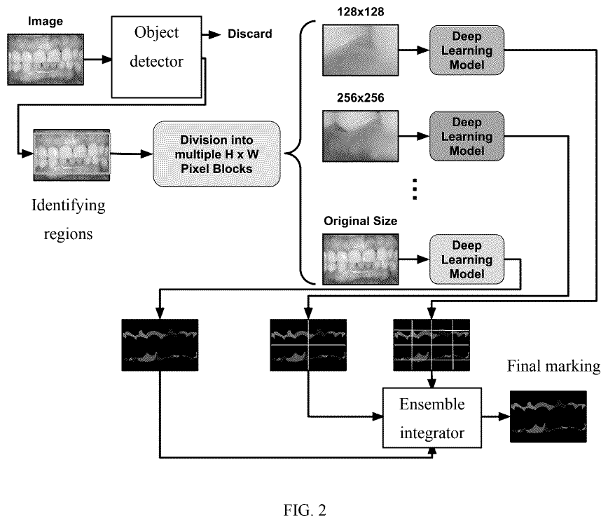

In the illustration presented in FIG. 2, a frontal view intraoral image undergoes an initial screening process with an object detector aimed at excluding unsuitable images, such as those with unclear gingival margins or insufficiently revealed gum areas. Following this screening, the object detector identifies a specific region of interest within the images, subsequently dividing this region into multiple pixel blocks with dimensions denoted as H×W, where H and W represent the block's height and width. This division can be performed in various sizes, constituting an independent process to generate pixel blocks of different dimensions, for instance, 128×128 and 256×256 pixel blocks. Subsequently, each of these pixel blocks, along with the original photograph, undergoes analysis by their corresponding deep learning models, generating distinct oral condition markings for each. Finally, all these individual markings are amalgamated through an ensemble integrator to produce a consolidated and conclusive final marking.

In accordance with a second aspect of the present invention, a method of processing intraoral images utilizing the aforementioned ML based image processing system is introduced.

The process initiates by acquiring one or more frontal view intraoral images through the image receiver. These images then undergo a filtering process using the object detector, which identifies regions in need of further processing in the frontal view intraoral images.

Following identification, the object detector segments these regions into pixel blocks, employing a precise approach. The target is to maintain a set of focused pixel blocks around the gum margins with a specific proportion observed in the local field of view. The segmented pixel blocks, alongside the original regions, are subject to detailed analysis and annotation facilitated by multiple autoencoders with deep neural networks. This can effectively solve the problems of scale inconsistency and data imbalance, thereby improving network performance. This comprehensive analysis aims to generate oral structure condition markings, considering parameters such as gum color, gingival margin smoothness, gingival margin curvature, and other relevant features contributing to the overall oral structure condition including texture features near the gingival margin such as stippling, swelling appearances.

The subsequent step involves integrating the annotated oral structure condition markings into a complete marking. This integration process is orchestrated by the ensemble integrator, harmonizing individual markings for a unified representation of the oral structure condition. In some embodiments, the resulting complete marking includes annotations for a healthy gingival margin, a questionable gingival margin, and a diseased gingival margin. These annotations serve as indicators of oral health status, prominently marked on the frontal view intraoral images.

The analysis and annotation process delves into parameters related to gum color, gingival margin smoothness, gingival margin curvature, and potentially other factors crucial for a comprehensive assessment.

Each type of gingival margin annotation is associated with different characteristics. A healthy gingival margin is marked by a pink gum, a smooth gingival margin, and the absence of bleeding spots. A questionable gingival margin is characterized by a transition to red gum color, a rough gingival margin, or gum swelling. Conversely, a diseased gingival margin is indicated by white or red patches on the gum, generalized redness, ulcers, gum swelling, or bleeding spots.

Similarly, the frontal view intraoral images used in this method display at least 3 mm of gingival tissue from both maxillary and mandibular gingival margins. Additionally, a clear gingival margin between the gum and teeth ensures standardized representation for accurate image processing.

Basically, the method commences with the acquisition of frontal view intraoral images capturing both teeth and gum from the subject. These images are then applied to the autoencoders. The autoencoders, designed to identify specific regions within the images, employs a segmentation technique, breaking down these regions into pixel blocks for detailed examination. The analysis involves a comprehensive review of parameters such as the colors of the gum, the smoothness and curvature of the gingival margin, among others. Subsequently, the autoencoders generates precise markings denoting gingivitis areas.

Building upon this, the autoencoders undergo meticulous training, leveraging standard frontal view intraoral images assessed by a qualified dentist. The dentist, serving as the reference expert, meticulously marks all the gingival margins displayed in these standard images, categorizing them into healthy, questionable, or diseased states. The autoencoders's training is designed to analyze a spectrum of parameters, including the colors of the gum, the smoothness and curvature of the gingival margin, and other relevant features.

### EXAMPLES

### Example 1

The following experiments were approved by the Institutional Review Board of the University of Hong Kong/Hospital Authority Hong Kong West Cluster (HKU/HA HKW IRB), Hong Kong Special Administrative Region, China (reference numbers: UW 20-230 and UW 21-447), and the Research, Ethics/Safety Sub-Committee (RESS) of Hong Kong Chu Hai College, Hong Kong Special Administrative Region, China (reference number: RESS/2022/06/006).

Consecutive participants were recruited amongst patients attending the Comprehensive Dental Clinic of the University Dental Hospital from 2020 to 2022 according to the selection criteria (Table 1). Informed consent was obtained from all participants. Frontal-view intraoral images were taken using a digital single lens reflex (SLR) camera (EOS 700D, Canon) with a macro lens (EF 100 mm f/2.8, Canon) and a ring flash (Marco Ring Lite MR-14EX, Canon).

TABLE 1
Inclusion and exclusion criteria of participants

| | |
|---|---|
| Inclusion criteria | Participants who are Chinese and aged 18 or older |
| | Participants who are able to give consent |
| | Participants who have 5 or more anterior teeth |
| | Participants who have adequate mouth opening for |
| | visualization of at least 3 mm gingival tissue from maxillary |
| | and mandibular gingival margins |
| | Participants who are able to attend dental appointment and |
| | hold still during taking intraoral photograph |
| Exclusion criteria | Participants who have non-plaque oral mucosal diseases that |
| | preclude the use of mirror retractors |

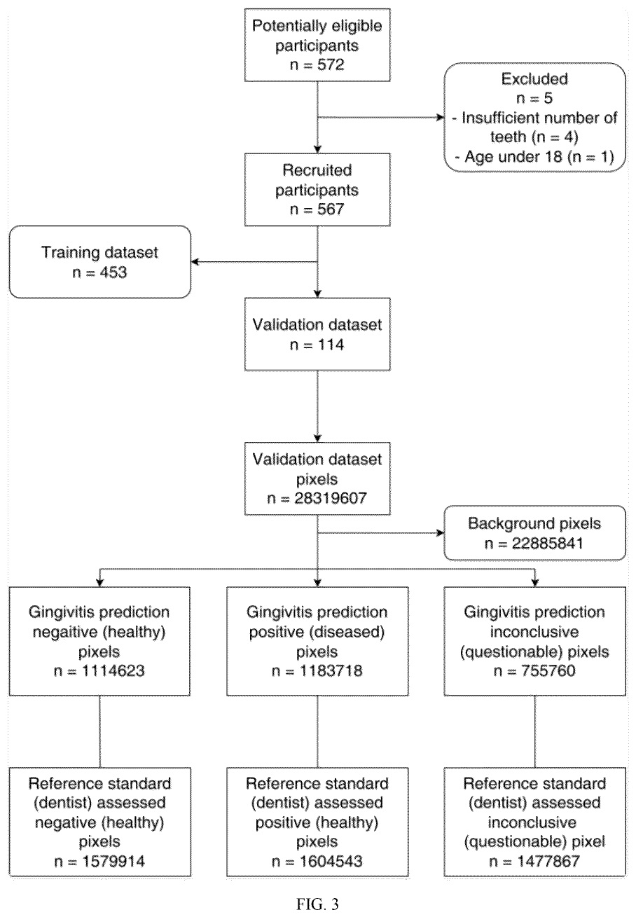

As shown in FIG. 3, 572 potential participants were screened according to the study criteria. Four were rejected due to insufficient number of anterior teeth, and one was rejected due to age younger than 18 years. The number of recruited study participants was 567. A total of 567 frontal-view intraoral images were taken from the study participants. Amongst the collected images, around 80% of the total (n=453) were designated as training datasets, and the rest (n=114) were designated as validation datasets.

The gingival conditions of all the collected intraoral images were labelled by a calibrated assessor, who was a dentist and based on visual assessment on a computer monitor. The areas of interest within each frontal images were the gingival margin and around 3 mm gingival tissues apical to the margin. These areas were classified into 1 of 3 categories: healthy, diseased, or questionable, based on a screening instrument, Oral Health Assessment Tools (OHATs), where the definitions were as follows:
Healthy: pink, smooth, no bleeding;
Questionable: red, rough, swollen; and
Diseased: white/red patches, generalized redness, ulcers, swollen, bleeding.

Unlabeled areas were classified in the system as background, making a total of 4 classifications. One week later, 10% of all images were labelled again by same assessor to measure the intra-assessor reliability in diagnosis of gingival conditions healthy, diseased, or questionable. The kappa value of the assessor was measured.

Around 450 images were randomly designated as training datasets by randomization table, and the rest of the images were designated as validation datasets. Images of the training datasets were augmented by cropping, rotating, or flipping randomly to enhance the training quality.

The training datasets consisted of 113,745,208 pixels in total, with U.S. Pat. Nos. 9,270,413; 5,711,027; and 4,596,612 pixels labelled as healthy, diseased, and questionable, respectively. The validation datasets consisted of 28,319,607 pixels in total, with U.S. Pat. Nos. 1,579,914; 1,604,543; and 1,477,867 pixels labelled as healthy, diseased, and questionable, respectively. The assessor had a kappa value 0.92 over 2 attempts of labelling, which indicated high reliability.

Images from the training datasets were input into the AI system for training. After training, the AI system was then instructed to diagnose the gingival status of intraoral images of the validation datasets. Both the training and validation processes were performed on a Linux system powered by a graphic card of NVIDIA GeForce RTX 3090. The batch number was set as 4, which is the number of classifications, and the number of training iterations was set to be 30,000, a common iteration number to train 2-dimension AI systems.

The performance of the AI system was measured by true-positive rate, true-negative rate, false-positive rate, and false-negative rate. True-positive rate was the outcome where the AI correctly detected the diseased status, and true-negative rate was the outcome where the AI correctly detected the healthy status. False-positive rate and false-negative rate were the outcomes where AI treated healthy sites as diseased and diseased sites as healthy, respectively. Sensitivity and specificity were calculated based on the following formula:

$$Sensitivity = TruePositive / ( TruePositive + FalseNegative )$$

$$Specificity = TrueNegative / ( TrueNegative + FalsePositive )$$

Mean intersection-over-union, a ratio of true predictions (positive and negative) against the ground truth (actual health status), was a wide-adapted performance metric for segmentation models in field of artificial intelligence and was calculated by dividing the sum of 4 intersection-over-unions of healthy, diseased, questionable, and background by 4. Intersection-over-union of each category was calculated by the following formula:

$$Intersection - Over - Union = ( α ⋂ β ) / ( α ⋃ β ) ;$$

where α was the dataset of diagnosis by dentist and β was the dataset of prediction by the AI system. In mathematics, the symbol ∪ represents the union of 2 sets, whilst ∩ represents the intersection of the sets. Accuracy ranged from 0.00 to 1.00, and 1.00 is considered to be the maximum accuracy. The common threshold for acceptable prediction is 0.50.

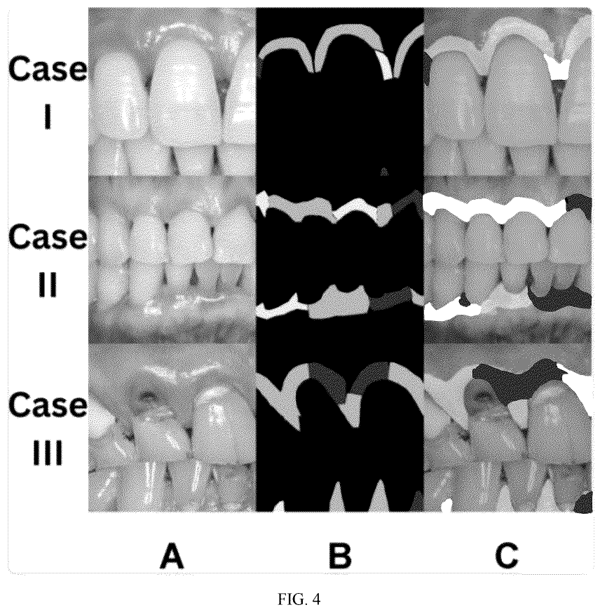

As presented in FIG. 4, AI correctly predicted 1,114,623 healthy and 1,183,718 diseased pixels (Table 2), with a sensitivity of 0.92 and a specificity of 0.94. The mean intersection-over-union was 0.60.

TABLE 2
Predictions of the AI system compared to the diagnosis of a calibrated dentist.

| | | Predictions of the system (pixels) | | | |
|---|---|---|---|---|---|
| | | Predicted as | Predicted as | Predicted as | Predicted as |
| | | healthy | diseased | questionable | background |
| Diagnosis | Diagnosed | 1,114,623 | 72,048 | 140,694 | 252,549 |
| of dentist | healthy | | | | |
| (pixels) | Diagnosed | 93,017 | 1,183,718 | 76,597 | 251,211 |
| | diseased | | | | |
| | Diagnosed | 248,694 | 258,035 | 755,760 | 215,378 |
| | questionable | | | | |
| | Diagnosed | 275,697 | 352,303 | 143,442 | 22,885,841 |
| | background | | | | |

The results evidence that the AI system, after training with adequate number of intraoral images, is able to predict the gingival health status with accuracy, in terms of sensitivity and specificity, at or above 0.90. The novel AI system is able to identify specific sites with and without gingival inflammation with sensitivity and specificity that are almost on par with human dentists, which is one of the current methods used to detect gingival inflammation clinically. The result is encouraging and supported the use of AI in detection of gingivitis on intraoral images.

It is worth noting that when a population has a high prevalence of a particular disease such as gingivitis, it is expected that its diagnostic tests usually have high sensitivity, that is, a positive result when there is a disease, and low specificity, that is, a negative result when there is no disease. This is because it is easier for a diagnostic test to detect a disease when it has high prevalence and vice versa. However, gingivitis is a site-specific disease, and healthy sites may be found in patients with gingivitis. Therefore, similar numbers of healthy and diseased pixels as well as similar levels of sensitivity and specificity are found in the above results. However, with training datasets in larger quantities as well as in decreased diversity, the training outcomes may be further improved.

The functional units and modules of ML based image processing system and methods in accordance with the embodiments disclosed herein may be embodied in hardware or software. That is, the claimed image processing system may be implemented entirely as machine instructions or as a combination of machine instructions and hardware elements. Hardware elements include, but are not limited to, computing devices, computer processors, or electronic circuitries including but not limited to application specific integrated circuits (ASIC), field programmable gate arrays (FPGA), microcontrollers, and other programmable logic devices configured or programmed according to the teachings of the present disclosure. Computer instructions or software codes running in the computing devices, computer processors, or programmable logic devices can readily be prepared by practitioners skilled in the software or electronic art based on the teachings of the present disclosure.

The ML based image processing system may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can include, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

In some embodiments, the ML-based image processing system further includes a display and/or a user interface, which may encompass a variety of devices, including but not limited to smartphones, tablets, laptops, desktop computers and dedicated medical equipment interfaces. These devices provide various form factors and screen sizes to accommodate different user preferences and usage scenarios and ensures accessibility across different platforms. Furthermore, these devices facilitate user interaction and visualization of the processed intraoral images, offering convenient and intuitive means for healthcare professionals to analyze and interpret the results. Additionally, the inclusion of such displays and user interfaces enhances accessibility and usability, allowing for seamless integration into existing workflows and clinical environments.

The ML based image processing system may also be configured as distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A machine learning (ML) based image processing system for image processing of intraoral images comprising:
an image receiver for processing one or more frontal view intraoral images of intraoral structures;
an object detector that filters frontal view intraoral images, identifies regions for further image processing in the frontal view intraoral images, and segment the regions into pixel blocks;
multiple autoencoders with multiple deep neural networks for image analysis and image annotation of the regions and the segmented pixel blocks for generating oral structure condition markings; and
an ensemble integrator that integrates the oral structure condition markings to a complete marking.

2. The system of claim 1, wherein the multiple autoencoders using deep neural networks corrects intraoral images by image analysis that corrects the frontal view intraoral images containing blurriness caused by body movement or scaling and performs image color balance.

3. The system of claim 1, wherein the segmentation of the regions of interest is able to be performed multiple times to generate different sets of the pixel blocks in different sizes, so as to increase the accuracy of the marking.

4. The system of claim 1, wherein the frontal view intraoral images show at least 3 mm gingival tissue from maxillary and mandibular gingival margins and a clear gingival margin between the gum and teeth.

5. The system of claim 1, further comprising a user interface and a display for displaying the complete marking.

6. The system of claim 1, further comprising a memory unit for storing a database of annotated intraoral images, wherein the multiple autoencoders are trained on this database to improve the accuracy and efficiency of the image analysis.

7. A method of processing intraoral images utilizing the machine learning (ML) based image processing system of claim 1, comprising:
acquiring one or more frontal view intraoral images of intraoral structures by the image receiver;
filtering the frontal view intraoral images and identifying regions thereof for further image processing in the frontal view intraoral images utilizing the object detector;
segmenting the regions into pixel blocks by the object detector;
analyzing and annotating the regions and the segmented pixel blocks utilizing the multiple autoencoders with multiple deep neural networks for generating structure condition markings; and
integrating the oral structure condition markings to a complete marking by the ensemble integrator.

8. The method of claim 7, wherein the complete marking comprises annotations of a healthy gingival margin, a questionable gingival margin and a diseased gingival margin marked on the frontal view intraoral images.

9. The method of claim 7, wherein the step of analyzing and annotating the regions and the segmented pixel blocks comprises analyzing parameters comprising the colors of the gum, the smoothness of the gingival margin, the curvature of the gingival margin, texture features of the gum near the gingival margin such as stippling, swelling appearances.

10. The method of claim 9, wherein the texture features of the gum near the gingival margin comprise stippling and swelling appearances.

11. The method of claim 8, wherein the healthy gingival margin indicates that the gum is pink, the gingival margin is smooth and there is no bleeding spot on the gum; the questionable gingival margin represents that the gum turns red, the gingival margin is rough or the gum is swollen; and the diseased gingival margin indicates that there are white/red patches on the gum, the gum is generalized redness, there is ulcer on the gum, the gum is swollen or there is bleeding spot on the gum.

12. The method of claim 7, wherein the multiple autoencoders with multiple deep neural networks are trained with standard frontal view intraoral photographs assessed by at least one qualified dentist, wherein the qualified dentist marks the gingival margins displayed in the standard frontal view intraoral photographs and denotes them as healthy, questionable and diseased.

13. The method of claim 7, wherein the frontal view intraoral images show at least 3 mm gingival tissue from maxillary and mandibular gingival margins and a clear gingival margin between the gum and teeth.
